(19) United States
(12) Patent Application Publication
(10) Pub. No.: US 2022/0061971 A1
(43) Pub. Date: Mar. 3, 2022

(54) SYSTEM AND METHOD FOR DENTAL VENTILATION

(71) Applicant: Michael Cusack, Durango, CO (US)

(72) Inventor: Michael Cusack, Durango, CO (US)

(21) Appl. No.: 17/464,664

(22) Filed: Sep. 1, 2021

Related U.S. Application Data
(60) Provisional application No. 63/073,498, filed on Sep. 2, 2020.

(51) Int. Cl. A61C 17/02; A61C 17/06

(52) U.S. Cl. CPC A61C 17/0208; A61C 17/084

(57) ABSTRACT

A system and method for dental ventilation is provided including a fan mount assembly wherein a fan unit having a perimeter fan ring is engaged with the fan mount assembly by engaging a locking ring to the fan mount assembly over the perimeter fan ring. The fan unit is connected to an air intake using a duct hose. The air intake includes a light receptacle receiving a light to illuminate a patient and a battery holder receiving a battery to electrically power the light.

### CROSS-REFERENCE TO RELATED APPLICATIONS

The present application claims the benefit of U.S. Provisional Application No. 63/073,498, filed Sep. 2, 2020, entitled “System and Method for Dental Ventilation”, which is hereby incorporated by reference in its entirety.

### BACKGROUND OF THE INVENTION

The present invention generally relates to a ventilation unit. More particularly, the present invention relates to a dental ventilation unit with an improved air intake and fan mount assembly.

Transmission of communicable diseases can be a concern in a dental operatory, especially the transmission of SARS-CoV-2, which may result in COVID-19. SARS-CoV-2 is transmissible in at least two ways in a dental operatory. First, it may be transmitted during procedures utilizing high speed handpieces and ultrasonics through the generation of aerosols. Theses aerosols consist of mainly water and saliva. Virus particles shed from the upper respiratory tract may be attached to droplets of water and saliva which are then expelled as aerosols through air forced out of the conventional air driven handpiece. The SARS-CoV-2 virus may then be inhaled into the upper respiratory tract by the dental staff and infect them.

In addition, SARS-CoV-2 may also be transmitted in non-aerosol generating procedures. Research has shown that prolonged exposure to a virus carrier in a poorly ventilated area is likely to lead to contracting SARS-CoV-2. This may occur as a result of what is known as airborne transmission. As respiratory droplets are expelled through normal speaking and breathing the water content begins to evaporate, resulting in an extremely light particle with attached virus particles that may be able to remain aloft for a considerably long period. Recent scientific studies were unable to find a biologic half-life of the SARS-CoV-2 artificially suspended in a rotating drum at 16 hours, which was the extent of the allotted experimental time. As a result, the virus may be transmitted from patient to patient, or patient to staff by inhaling virus particles that were generated from previous appointments and remain aloft in the dental operatory. Due to the hardiness of the SARS-CoV-2 virus, airborne suspended virus particles from a morning patient may potentially be transmitted to anyone having a subsequent appointment in the same room.

### BRIEF SUMMARY OF THE INVENTION

One or more of the embodiments of the present invention provide a dental ventilation system and method. The dental ventilation system includes an improved fan mount assembly and several embodiments of an air intake. The fan mount assembly receives a fan unit having a perimeter ring in a fan unit aperture so that the perimeter ring is positioned on top of and supported by the top surface of the fan mount assembly. A locking ring is then positioned over the perimeter ring and bolts are passed through bolt holes in the locking ring and fan mount assembly to secure the fan unit and locking ring to the fan mount assembly. A duct hose connects the fan mount assembly to an air intake and several embodiments of the air intake are provided. In one embodiment, the air intake includes a light receptacle positioning a removable light to illuminate a patient and a battery holder holding a removable battery that is electrically connected to and powers the removable light. The air intake also includes a plurality of barbs that engage the air intake with the duct hose.

### BRIEF DESCRIPTION OF THE DRAWINGS

FIG. 1 illustrates an exploded view of an embodiment of a fan mount assembly according to an embodiment of the present invention.

FIG. 2 shows the assembled fan mount assembly with the fan unit installed.

FIG. 3 illustrates the fan mount in a flat, pre-folded state, according to an embodiment of the present invention.

FIG. 4 illustrates an underside view of the fan mount of FIG. 3 in its assembled state.

FIG. 5 illustrates an exploded view of an embodiment of an air intake according to an embodiment of the present invention.

FIG. 6 illustrates the air intake of FIG. 5 in an assembled view.

FIG. 7 illustrates an alternative embodiment of the air intake fixture.

FIG. 8 illustrates a lower view of the fan mount assembly of FIG. 1 with a duct positioned over the lower aperture of the fan unit.

FIG. 9 illustrates an alternative integrated air intake.

FIG. 10 illustrates an embodiment of a dental ventilation system positioned in a dental operatory according to an embodiment of the present invention.

FIG. 11 illustrates an alternative embodiment for an air intake that allows the attachment of a removable light and battery pack.

FIG. 12 illustrates one embodiment of the battery holder.

FIG. 13 illustrates one embodiment of the light receptacle as seen from the top side of the air intake.

FIG. 14 illustrates one embodiment of the bilateral barbs to engage grooves in the air intake hose.

FIG. 15 illustrates a view of the air intake from underneath.

FIG. 16 shows a cut-away view of the intake edges of the air intake that include a circumferential grip section.

FIG. 17 illustrates a side view of the air intake of FIGS. 11-16 installed in a dental ventilation system positioned in a dental operatory.

FIG. 18 illustrates a view from underneath the air intake of FIGS. 11-17 installed in a dental ventilation system positioned in a dental operatory.

### DETAILED DESCRIPTION OF THE INVENTION

One or more embodiments of the present dental ventilation system and method were designed with the intent of disease control. Specifically the present dental ventilation system is designed to reduce the transmission of SARS-CoV-2 in the dental operatory.

One or more embodiments of the present ventilation system are designed to provide an air intake as close as possible to the patient in order to evacuate aerosols generated by the patient to the outdoors where the aerosols are diluted or passed through a HEPA (High Efficiency Particulate Air) filter and subsequently into the return plenum of the HVAC (Heating, Ventilation and Air Conditioning) system.

FIG. 10 illustrates an embodiment of a dental ventilation system 1000 positioned in a dental operatory 1005 according to an embodiment of the present invention. As shown in FIG. 10, the dental ventilation system 1000 includes an air intake 1010 including hose clamps 1020 to connect the air intake 1010 to a duct hose 1030. The duct hose 1030 may be connected to a filter box 1040. Air may further flow from the filter box 1040 through a fan input conduit 1050 to an inline fan 1060. The inline fan 1060 may be connected to the fan input conduit 1050 and fan output conduit 1080 using tube clamps 1070. Additionally, as further describe below, a pressure gauge 1090 may be positioned in the dental operatory 1005 and may adjust the speed of the inline fan 1060 to maintain a desired room pressure.

As further described below, the air intake 1010 may be configured with a bellmouth design and may include handles and lights positioned to illuminate the patient, for example for operation. The filter box 1040 may include a HEPA filter and/or ultraviolet (UV) sanitation system. Additionally, as further described below, the inline fan 1060 may employ a clamshell mount that may provide additional stability and as tight an air-tight connection as possible. The fan output conduit 1080 may vent to a roof vent or an return plenum, for example with a HEPA filter. The pressure gauge 1-9—may be a magnahelic differential pressure gauge, for example if negative pressure in the operatory 1005 is desired.

In operation, the present ventilation system reduces the incidence of aerosols in the dental operatory by providing what is referred to as air changes per hour. Basically, air change is described as the time it takes to remove a room's entire volume of air. One or more embodiments of the present system are engineered to provide at least twelve air changes per hour per the ASHRAE (American Society of Heating, Refrigerating and Air-Conditioning Engineers) specifications for an airborne isolation room in a typical dental operatory. The typical dental operatory consists of approximately 1000 cubic feet. In one embodiment, the present system is designed to flow at least 200 cubic feet a minute, but can be adjusted to flow more or less based on the room size to a maximum of 1000 cubic feet per minute. At 200 cubic feet a minute, a typical 1000 cubic feet room will undergo an air change in 5 minutes, resulting in the isolation room specification of twelve air changes an hour. At this air exchange rate the CDC (Center for Disease Control) states the room to be 99% free of airborne contaminants in 23 minutes and 99.9% free in 35 minutes. This, the present system provides a safe environment in 30 minutes for subsequent patients and staff.

In regards to the airflow, in one embodiment the fans have a rheostat to control the speed, which may be controlled by a small dial in the wall like a light dimmer. One specific embodiment is the WC15 from Fantech. In one embodiment, the amount of air flowed may be dependent on the makeup air system, which preferably provides at least 200 cfm (cubic feet per minute) to be effective at aerosol removal, but may flow more to increase aerosol removal. Air removed from the operatory is replaced by the makeup air system to maintain proper building pressurization. The fan speed may be set using the rheostat by the mechanical contractor based on room size, desired air flow (at least 200 cfm), and make up air system.

In one embodiment, the present system is designed to utilize a sealed inline fan motor. This type of motor is less expensive and much quieter than the typical vacuum blower type motors. The prototype unit was built using the Fantech fg6x1 unit. In order for the fan to function properly six inch duct was chosen to keep the static pressure exerted on the fan within the manufacturer's specifications. However, different fans may be utilized and ductwork of other specifications may be utilized depending on the fan.

The exhaust portion of the system may include anything downstream of the fan. In a direct exhaust application the fan is connected to a roof duct directly with a flexible duct section secured on each end with a metal band type clamp typical of ductwork.

When direct exhaust system is not possible due to climate and make up air requirements the system may be connected to the return plenum of the HVAC system. Such an application will use the placement of a filter system between the intake duct and fan unit. Currently HEPA type filters are currently used to filter the 0.125 micron Sars-Cov-2 virus. HEPA filters were originally designed to filter radioactive dust and do a great job of filtering particles in 0.1-0.3 micron range.

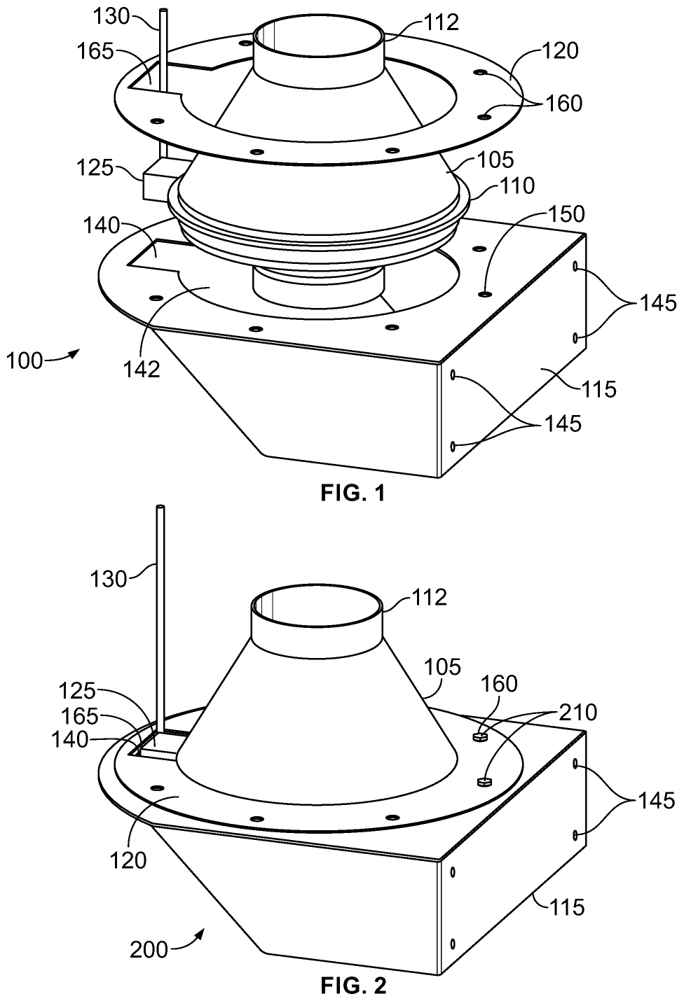

FIG. 1 illustrates an exploded view of an embodiment of a fan mount assembly 100 according to an embodiment of the present invention. The fan mount assembly 100 includes a fan unit 105 having a fan ring 110 and a fan exhaust 112, a fan mount 115, and a locking ring 120. The fan unit 105 is also connected to a power box 125 which is supplied power by a power cord 130. The fan mount 115 includes a fan mount power box keyhole aperture 140, a fan unit aperture 142, a plurality of structural element mounting holes 145 and a plurality of fan mount bolt holes 150. The locking ring 120 includes a plurality of locking ring bolt holes 160 and a locking ring power box keyhole aperture 165.

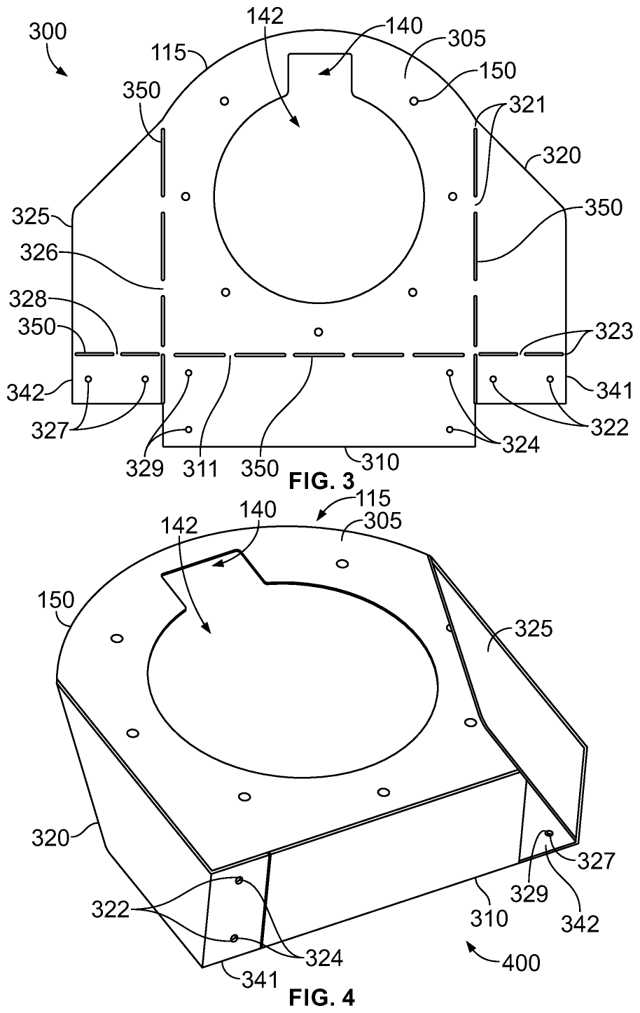

In operation, the fan mount assembly 100 a fan ring 110 that rests on the upper surface of a fan mount 115. The fan mount 115 is shown in FIGS. 3 and 4 in a collapsed view and an assembled view, as discussed below. The fan mount 115 includes the power box keyhole aperture 140 which provides an aperture for accepting the power box 125 once it is electrically connected to the fan unit 105. Additionally, the structural element mounting holes 145 are positioned on a vertical side element of the fan mount 115 and bolts or screws may be passed through the structural element mounting holes 145 to engage the fan mount 115 with one or more structural elements. Additionally, the fan mount 115 includes the fan mount bolt holes 150 positioned in the horizontally-oriented top surface of the fan mount 115. Further, the fan unit aperture 142 is sized to receive the bottom structure of the fan unit 105 through the top surface of the fan mount 115 while being small enough to prevent the fan ring 110 from passing through the top surface of the fan mount 115.

In operation, once the fan unit 105 has been positioned in the fan mount 115 to that the fan ring 110 is resting on the top surface of the fan mount 115, the locking ring 120 is placed over the fan ring 110. In this position, the power box 125 is located in the fan mount power box keyhole aperture 140 and the locking ring power box keyhole aperture 165. Additionally, the locking ring bolt holes 160 and fan mount bolt holes 150 are aligned so that a bolt may be passed vertically through each of the aligned bolt holes in order to clamp the locking ring 120 to the fan mount 115 thereby fixing the fan unit 105 in place in the fan mount 115.

FIG. 2 shows the assembled fan mount assembly 115 with the fan unit 105 installed. As shown, bolts 210 are passed through the locking ring bolt holes 160 bolt in the locking ring 120 and the fan mount bolt holes 150 in the upper surface of the fan mount 115 to secure the fan unit 105 to the fan mount 115. Additionally, the fan mount power box keyholes aperture 140 in the fan mount 105 and locking ring power box keyhole aperture 165 in the locking ring 120 accommodate the power box 125 of the fan unit 105 and its power cord 130. In operation, as shown above, additional ducting is attached to the fan exhaust 112 in order to direct the exhaust to a HEPA filer or an external vent.

In another embodiment, to promote the air seal integrity of the Fantech fan unit a clamshell type mount is employed, as further described below. In this embodiment, the fan unit has a small ring or lip resulting from the joining and clamping of the two halves, upper and lower, of the galvanized steel fan housing. The fan is placed in the clamshell fan mount and rests on the ring. Then the securing piece with aligning geometry is placed over the fan housing on top of the ring on the fan. The securing piece is hinged at one end, allowing the securing piece to rotate up and down like a clamshell.

The securing piece is then bolted to the lower fan mount, which clamps the fan in place. The clamping mount is used in order to maintain the air sealed quality of the fan unit by eliminating the use of sheet metal screws placed into the housing in the typical mounting fashion. The clamping mount is mounted above the operatory. The fan has a lower intake ring designed to accommodate the appropriate size duct. In this application it is a 6″ duct.

FIG. 3 illustrates the fan mount 115 in a flat, pre-folded state 300, according to an embodiment of the present invention. As shown in FIG. 3, the fan mount 115 includes a main fan mount section 305, rear flap 310, a first side flap 320, a second side flap 325, a rear flap fold line 311, a first side flap fold line 321, a second side flap fold line 326, first side flap mounting holes 322, a first side flap rear segment 341, a second side flap rear segment 342, second side flap mounting holes 327, a first side flap bottom fold line 323, a second side flap bottom fold line 328, first side rear flap mounting holes 324, second side rear flap mounting holes 329, fan mount bolt holes 150, a fan unit aperture 142, and a fan mount power box keyhole aperture 140.

As shown in FIG. 3, the rear flap fold line 311, first side flap fold line 321, second side flap fold line 326, first side flap bottom fold line 323, and second side flap bottom fold line 328, have been fabricated to include a plurality of slots 350. The inclusion of the slots 350 allows the fan mount 115 to be easily folded into its desired wedge shape.

In operation, to assemble the fan mount 115, the first side flap 320 is bent along the first side flap fold line 321 so that the first side flap 320 is perpendicular to the main fan mount section 305. Similarly, the second side flap 325 is bent along the second side flap fold line 326 so that the second side flap 325 is perpendicular to the main fan mount section 305. Next, the first side flap rear segment 341 is bent along the first side flap bottom fold line 323 so that the first side flap rear segment 341 is perpendicular to the first side flap 320. Similarly, the second side flap rear segment 342 is bent along the second side flap bottom fold line 328 so that the second side flap rear segment 342 is perpendicular to the second side flap 325. Next, the rear flap 310 is bent along the rear flap fold line 311 so that the rear flap 310 is perpendicular to the main fan mount section 305. By bending the rear flap 310 into this configuration, the first side flap mounting holes 322 align with the first side rear flap mounting holes 324 and the second side flap mounting holes 327 align with the second side rear flap mounting holes 329.

FIG. 4 illustrates an underside view of the fan mount 115 of FIG. 3 in its assembled state 400. As shown in FIG. 4, the fan mount 115 includes the main fan mount section 305, rear flap 310, the first side flap 320, the second side flap 325, the first side flap mounting holes 322, the first side flap rear segment 341, the second side flap rear segment 342, the second side flap mounting holes 327, the first side rear flap mounting holes 324, the second side rear flap mounting holes 329, the fan mount bolt holes 150, the fan unit aperture 142, and the fan mount power box keyhole aperture 140.

As shown in FIG. 4 and described above, the first side flap 320, second side flap 325, and rear flap 310 have been bent along their respective fold lines so that they are perpendicular to the main fan mount section 305. Additionally, the first side flap rear segment 341 and second side flap rear segment 342 have been bent along their respective fold lines so that they are perpendicular to their respective first side flap and second side flap. Additionally, the first side flap mounting holes 322 now align with the first side rear flap mounting holes 324 and the second side flap mounting holes 327, now align with the second side rear flap mounting holes 329.

In operation the aligned first side flap mounting holes 322, first side rear flap mounting holes 324, second side flap mounting holes 327, and second side rear flap mounting holes 329 form the plurality of structural element mounting holes 145 shown in FIG. 1.

Alternatively, FIG. 4 shows the fan mount folded into its wedge shape along the lines indicated by the pre-cut slots. As shown, the aligning mount holes on each side flap have been folded so that they are in line with the aligning mount holes on the rear flap of the fan mount. When the fan mount is engaged with a structural support, a bolt or screw is passes through the aligned mount holes of the side and rear flaps and then into the structural support. This engages the aligned mount holes of the side and rear flaps and maintains the structural stability of the fan mount.

A self-supporting pliable duct material, for example that known by the brand name Pliaduct, may extend into the operatory. The duct fully when extended terminates roughly 6″ from the oral cavity of a reclined patient. The pliable duct material allows the intake to be adjusted by an operator in 3 dimensions to any desired position to accommodate the patient's position and the line of vision needs of the dentist while positioning the intake as close as possible to the oral workspace in order to collect the maximum of aerosols generated. The pliable duct maintains the intake position after each adjustment due to its internal rigid structure, which is rigid enough to support the weight of the duct as well as the intake to the duct.

In addition, the pliable duct allows the intake be positioned above the patient when sitting to collect virus particles shed in normal breathing and speaking. It also allows the patient to be reclined without interference from the intake position. In one embodiment, the intake may be an aerodynamic shape such as a Bellmouth, as are used in turbine engine intakes. The intake may alternatively take various forms such as rectangular or ovoid.

As further shown and describe below, the outer surface of the intake slides into the inner portion of the duct. Small tabs on the intake engage slots prepared in the duct in order to connect the intake to the duct and to preventing the intake from slipping out of the duct and contacting the patient. In addition, small bolts or screws may be placed through the material of the duct and intake preventing the intake from sliding off. The intake and duct may alternatively or in addition be secured together in a friction arrangement with a metal band clamp and worm gear typical for ductwork. The intake may also have provisions for handles and the placement of a small spot type LED (Light-Emitting Diode) light for vision when the chair light is blocked by the intake.

In one embodiment, the light is mounted on the edge of the intake. The positioning and direction of the light may be adjustable independent from the intake. The beam width may vary between 4 and 20 degrees. The color of the light is preferably between 5000-7000 kelvin.

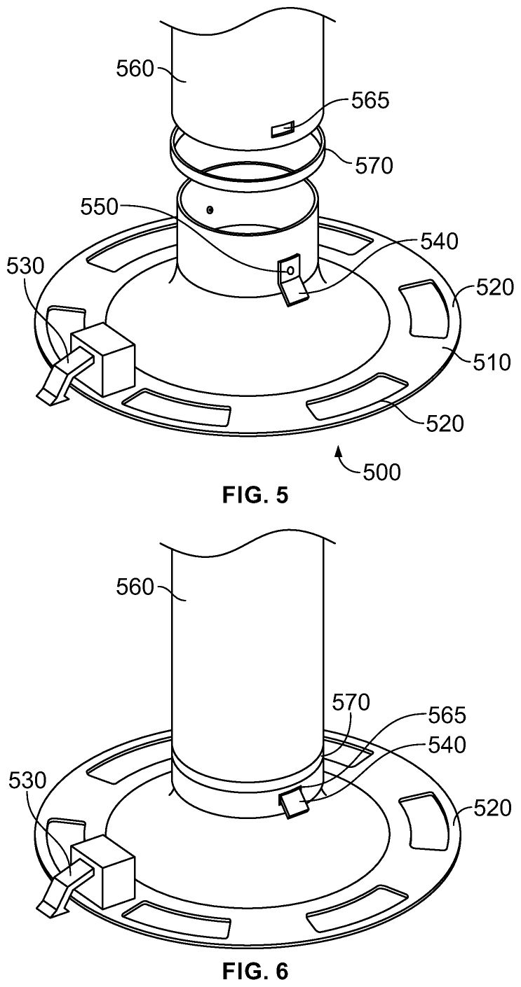

FIG. 5 illustrates an exploded view of an embodiment of an air intake 500 according to an embodiment of the present invention. As shown, the air intake includes an air intake fixture 510 having several handles 520 positioned around its radial extent. The air intake also includes a 530 light, a safety tab 540, and a rivet or bolt hole 550. The intake hose 560 for the duct includes an intake hose tab slot 565 that engages with the safety tab 540 of the air intake to mechanically engage the intake hose 560 with the air intake fixture 510. Additionally, a band clamp 570 is placed around the perimeter of the air intake hose 560 and then tightened in order to frictionally engage the air intake hose 560 with the air intake fixture 510. Additionally, a rivet or bolt may be passed through the air intake hose 560 and the rivet or bolt hole 550 of the air intake fixture 510 to further engage the air intake fixture 510 and air intake hose 560.

FIG. 6 illustrates the air intake of FIG. 5 in an assembled view. As shown, the safety tab 540 of the air intake fixture 510 has been engaged with the intake hose tab slot 565 and the band clamp 570 has been tightened around the perimeter of the air intake hose 560.

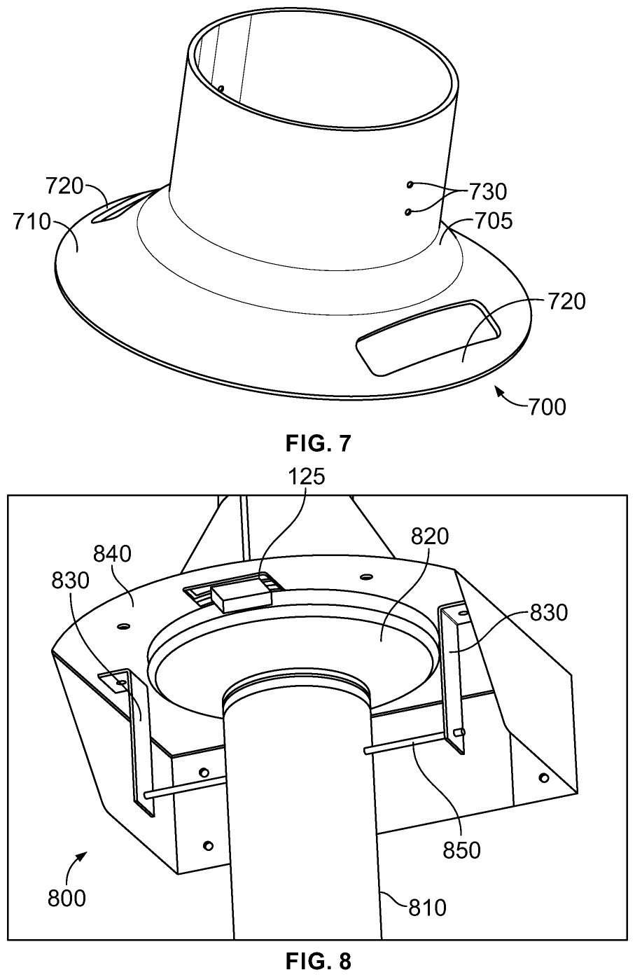

FIG. 7 illustrates an alternative embodiment 700 of the air intake fixture 705. As shown in FIG. 7, the intake edges 710 of the air intake fixture 705 nearest the patient are shaped in a conical segment or angled portion or horn-shaped portion. This provides for air intake over a wider area. As shown, the air intake includes a pair of handles 720 shaped as cutout apertures through the air intake fixture 705. Additionally, the holes 730 are shown for attachment of a safety tab similar to the safety tab 540 of FIG. 5.

FIG. 8 illustrates a lower view 800 of the fan mount assembly of FIG. 1 with a duct 810 positioned over the lower aperture of the fan unit 820. As shown in FIG. 8, a pair of brackets 830 have been attached to the lower portion of the fan mount 840. A safety pin 850 extends from a first of the pair of brackets 830 through the duct 810 and then terminates at the second of the pair of brackets 830. The brackets 830 and safety pin 850 through the duct 810 serve as a safety mechanism to keep the duct 810 attached to the lower portion of the fan mount 815.

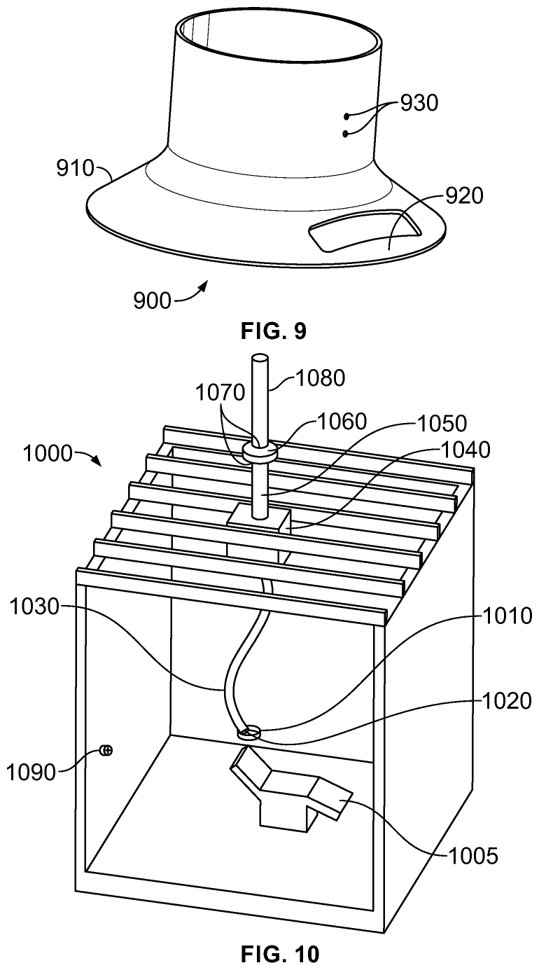

FIG. 9 illustrates an alternative integrated air intake 900. As shown in FIG. 9, the integrated air intake 900 includes a handle 920 and is generally angled or bell-shaped at the intake edges 910. Also shown are holes 930 for attachment of a safety tab similar to the safety tab 540 of FIG. 5.

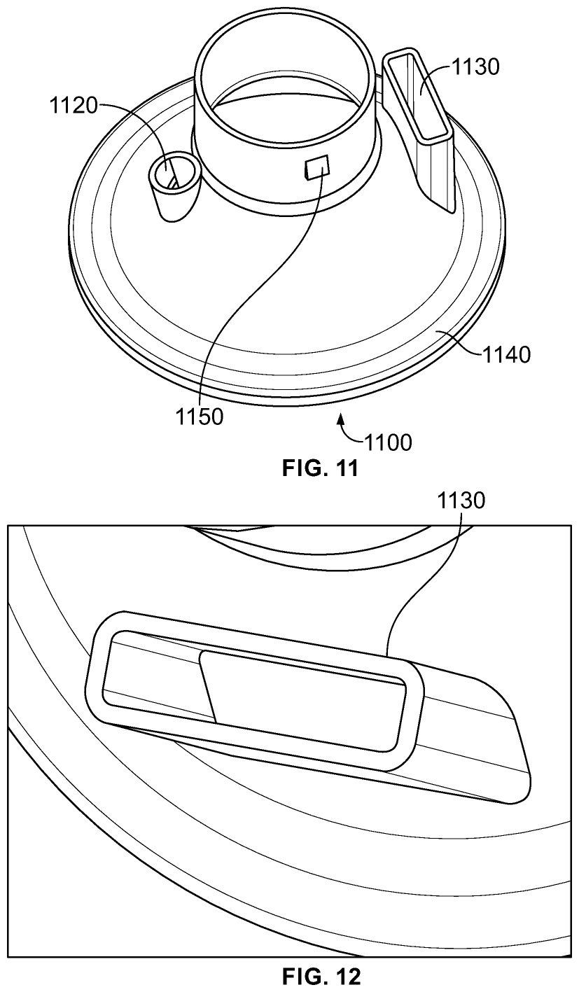

FIG. 11 illustrates an alternative embodiment for an air intake 1100 that allows the attachment of a removable light and battery pack. As shown in FIG. 11, the air intake 1100 includes a light receptacle 1120 for receiving a removable light and a battery holder 1130 for receiving a removable battery pack. Additionally, the air intake 1110 includes intake edges 1140 that include a circumferential grip and bilateral barbs 1150 to engage grooves in the air intake hose.

The alternative embodiment of the air intake 1100 of FIG. 11 may be optimized for 3D printing, for example, by removing the cut-out handles shown in other designs above and being shaped in a “Frisbee” shape for greater durability and ease of cleaning. Additionally, the light receptacle allows an operator to position a light so that the light source is inside the air intake 1100 and directed downward toward the patient. This provides an improved angle to view into the back of the oral cavity when the air intake is positioned 6-24 inches away from the patient. The air intake 1100 also includes the battery holder 1130 which is a compartment that is sized to fit a basic LED headlamp, the type often clipped to magnifying glasses. Additionally, there are barbs protruding where the intake hose attaches to engage the grooves to provide increased attachment between the air intake and the air intake hose. The barbs engage the spiral grooves in the air intake hose in a normal clockwise fashion to tighten, and counterclockwise to remove.

FIG. 12 illustrates one embodiment of the battery holder 1130. As shown in FIG. 12, the battery holder 1130 is a smooth, generally rectilinear compartment that terminates at the top surface of the air intake. In one embodiment, the battery holder may have a depth of 2.25 inches, a length of 2.5 inches, and a width of 0.5 inches. In operation, the batter pack may be slid into the battery holder 1130 and may be electrically attached to a light positioned in the light receptacle 1120 in order to power the light and illuminate the patient.

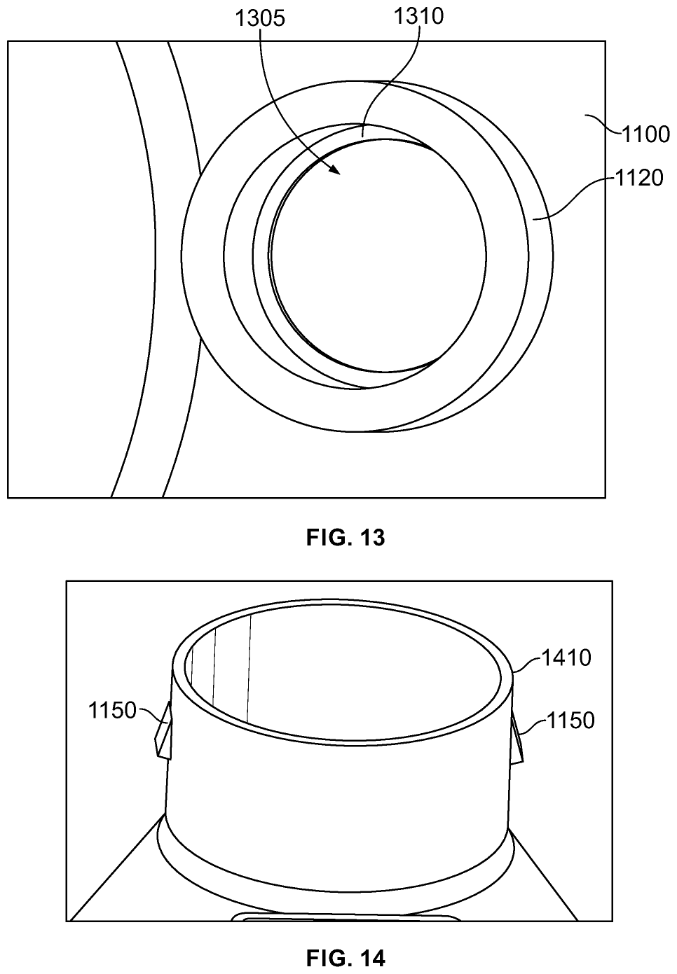

FIG. 13 illustrates one embodiment of the light receptacle 1120 as seen from the top side of the air intake 1100. As shown in FIG. 13, the light receptacle includes a generally cylindrical aperture 1305 extending through the light receptacle 1100. At the lower portion of the generally cylindrical aperture 1305 nearer the patient is a circular stop structure 1310. As shown in FIG. 13, in one embodiment the diameter of the generally cylindrical aperture 1305 is 0.85 inches and the width of the circular stop structure 1310 is 0.04 inches.

In operation, a generally cylindrical light is introduced into the light receptacle 1120 and slides downward into the light receptacle until the lowest edge of the light contacts the circular stop structure 1310. The light is electrically connected to a battery that is placed in the battery holder 1130 in order to power the light. The light may then be activated or deactivated by an operator so that light is emitted downward from the light receptacle and illuminates the patient.

FIG. 14 illustrates one embodiment of the bilateral barbs 1150 to engage grooves in the air intake hose. As shown in FIG. 14, the bilateral barbs 1150 are placed on opposite sides of the cylindrical structure 1410 forming the top aperture of the air intake 1100. In one embodiment, the bilateral barbs 1150 are 0.5 inches in height and extend 0.14 inches outward from the cylindrical structure 1410 forming the top aperture of the air intake 1100.

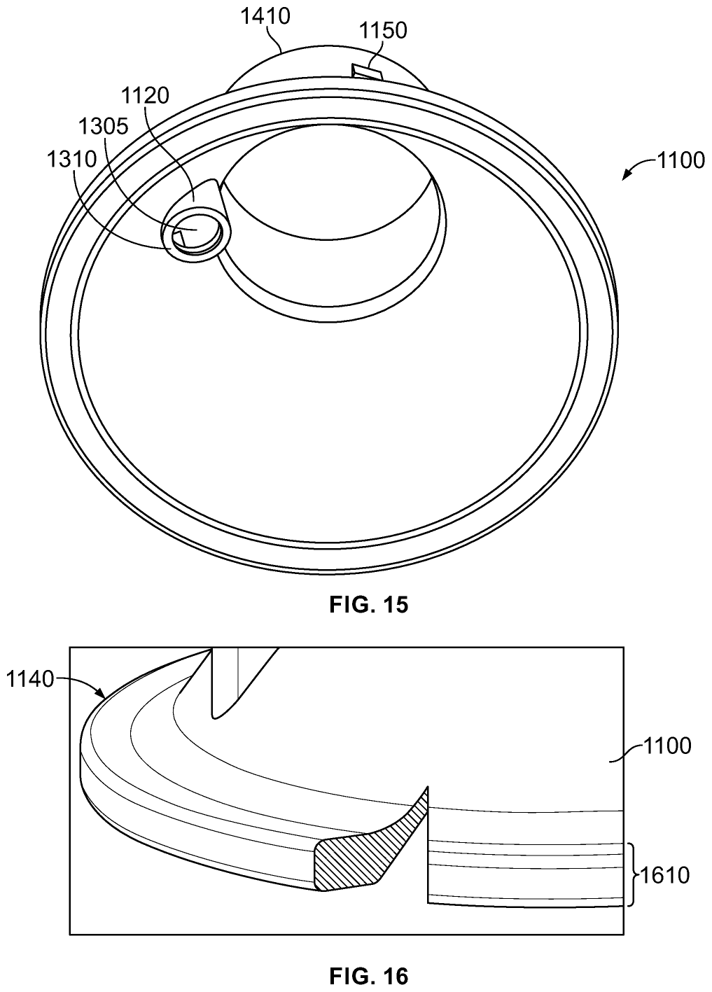

FIG. 15 illustrates a view of the air intake 1100 from underneath. As shown in FIG. 15, the light receptacle 1120 and its generally cylindrical aperture 1305 and circular stop structure 1210 are shown. Also shown is the cylindrical structure forming the top aperture of the air intake 1100 and one of the bilateral barbs 1150.

FIG. 16 shows a cut-away view of the intake edges 1140 of the air intake 1100 that include a circumferential grip section 1610. As discussed above, the circumferential grip section 1610 may be gripped by an operator to manually move the air intake 1100 and position and orientate it spatially as desired. Once moved to a certain position and orientation, the air intake remains stationary due to the stiffness of the duct hose 1030 connecting the air intake 1100 to the fan mount 115 or filter box 1040. As shown in FIG. 16, in one embodiment, the circumferential grip section 1610 may have a height of 0.5 inches and a thickness of 0.5 inches.

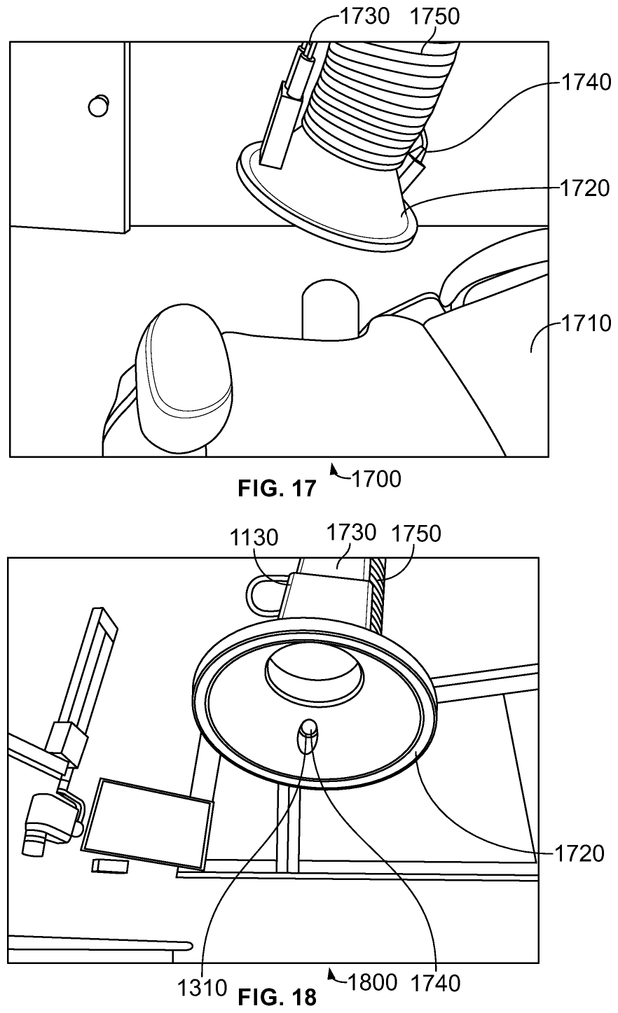

FIG. 17 illustrates a side view 1700 of the air intake of FIGS. 11-16 installed in a dental ventilation system positioned in a dental operatory. As shown in FIG. 17, a patient chair 1710 is positioned underneath the air intake 1720 and would typically be occupied by a patient. Additionally, as shown, the air intake 1720 includes a battery 1730 installed in the battery holder 1130 and a light 1740 installed in the light receptacle 1120 and electrically connected to the battery 1730. Additionally, the air intake 1120 is connected to the air intake hose 1750 by the bilateral barbs (not shown). Further, it can be seen that the light 1740 generates illumination in the location where a patient's mouth would typically be.

FIG. 18 illustrates a view 1800 from underneath the air intake 1720 of FIGS. 11-17 installed in a dental ventilation system positioned in a dental operatory. As shown in FIG. 18, the air intake 1720 includes the light 1740 installed in the light receptacle 1120 and resting against the circular stop structure 1310. Also shown are the battery holder 1130 and installed batter 1730 that is electrically connected to and powers the light 1740. The air intake hose 1750 connected to the air intake 1720 is also shown.

In one embodiment, the present invention bridges the gap between mechanical contractor and dentist. The present dental ventilation system has been dentist designed, tested, and streamlined for quick installation. All kits are tested with a NIST (National Institute of Standards and Technology)-calibrated air velocity probe prior to shipping, reducing time required to balance the system. It typically takes a single mechanical contractor one day per operatory, yielding a cost of $800-1500 per unit for installation.

One or more embodiments of the present dental ventilation system may provide everything required to turn any closed door dentist operatory into a negative pressure isolation room with direct outdoor exhaust and may also provide excellent particle capture and ventilation for open operatories. The present dental ventilation system has the airflow capacity for an isolation room and the capture velocity necessary for extra-oral particle vacuum. One or more embodiments of the present dental ventilation system includes an extra-oral suction intake with light, flexible intake duct hose that holds its position, commercial exhaust fan, stainless steel attic mounts, roof vent, ceiling trim ring, and all hardware.

Additionally, one or more embodiments of the present dental ventilation system may include a make up air system to keep proper building pressurization. The make up air system includes a filter for incoming air, lighted intake, flexible intake hose, commercial fan, ceiling trim ring, and stainless steel attic mounts and may be pre-calibrated with a NIST certified probe. Additionally, an inline duct heater or dehumidifier can be added depending on climate and current HVAC system.

One or more embodiments of the present dental ventilation system may also include HEPA filter units which may be identical to the direct exhaust units with the addition of a high quality inline HEPA filter unit. Preferable, only medical grade DOP (Dispersed Oil Particulate) tested HEPA filters are employed. In addition, the filter housing may include gauges indicating the filter health and may include attic mounting hardware with ducts routing the floor in the most distant corner for particle settling.

One or more embodiments of the present ventilation system may alternatively be used in more general surgical environment such as a hospital or doctor's office. Additionally, one or more embodiments of the present ventilation system may be employed in other settings such as a restaurant.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. A dental ventilation system including:
a fan mount assembly including:
a fan unit including a fan ring positioned around the perimeter of said fan unit;
a fan mount, wherein said fan ring is positioned proximal to said fan mount; and
a locking ring positioned over said fan ring and engaged with said fan mount to secure said fan unit to said fan mount;
an air intake receiving air that is desired to be removed; and
a duct hose connected to said air intake and said fan mount assembly wherein said duct hose provides a fluid connection between said air intake and said fan unit so that said fan unit may induce air that is desired to be removed through said air intake to said fan unit.

2. The system of claim 1 wherein said fan mount includes at least one fan mount bolt hole.

3. The system of claim 2 wherein said locking ring includes at least one locking ring bolt hole.

4. The system of claim 3 wherein said locking ring is engaged with said fan mount by passing at least one bolt through said at least one fan mount bolt hole and said at least one locking ring bolt hole.

5. The system of claim 1 wherein said fan mount includes at least one structural element mounting hole.

6. The system of claim 1 wherein said fan mount includes a power box keyhole aperture to accommodate a power box providing power to said fan unit.

7. The system of claim 1 wherein said locking ring includes a power box keyhole aperture to accommodate a power box providing power to said fan unit.

8. The system of claim 1 wherein said air intake includes at least one handle.

9. The system of claim 1 wherein said duct hose includes an intake hose tab slot.

10. The system of claim 9 wherein said air intake includes a tab that engages with said intake hose tab slot to engage said air intake and said duct hose.

11. The system of claim 1 wherein a band clam is placed around the perimeter of said duct hose to engage said duct hose with said air intake.

12. The system of claim 1 wherein said fan mount further includes a plurality of brackets exterior to said duct hose.

13. The system of claim 12 wherein a safety pin is positioned between said pair of brackets and through said duct hose to engage said duct hose with said fan mount.

14. The system of claim 1 wherein said air intake includes a plurality of barbs that engage said duct hose to connect said duct hose to said air intake.

15. The system of claim 1 wherein said air intake includes a light receptacle for receiving a light oriented to illuminate a patient.

16. The system of claim 15 wherein said light receptacle includes a cylindrical aperture extending through said air intake.

17. The system of claim 16 wherein said light receptacle includes a circular stop aperture to retain said light in said light receptacle.

18. The system of claim 15 wherein said air intake includes a battery holder for holding a battery.

19. The system of claim 18 wherein said battery is electrically connected to and powers said light.

20. A method for dental ventilation, said method including:
engaging a fan unit with a fan mount, wherein said fan unit included a fan ring positioned around the perimeter of said fan unit, including:
positioning said fan ring proximal to said fan mount;
positioning a locking ring over said fan ring; and
engaging said locking ring with said fan mount to engage said fan unit with said fan mount;
establishing a fluid connection between an air intake and said fan unit using a duct hose; and
inducing air that is desired to be removed though said air intake and said duct hose using said fan unit.
